(19) United States
(12) Patent Application Publication
(10) Pub. No.: US 2024/0266015 A1
(43) Pub. Date: Aug. 8, 2024

(54) ARTIFICIAL INTELLIGENCE SYSTEM FOR PROVIDING AUTOMATED CASE MANAGEMENT AND REPORTING

(71) Applicant: HELIX VIRTUAL MEDICINE, INC., BOCA RATON, FL (US)

(72) Inventors: Robert Rodriguez, Hillsboro Beach, FL (US); Elizabeth J. DeLong, Port St. Lucie, FL (US)

(73) Assignee: HELIX VIRTUAL MEDICINE, INC., BOCA RATON, FL (US)

(21) Appl. No.: 18/381,103

(22) Filed: Oct. 17, 2023

Related U.S. Application Data
(60) Provisional application No. 63/416,753, filed on Oct. 17, 2022.

(51) Int. Cl. G16H 15/00; G16H 20/13; G16H 40/20; G16H 50/20; G16H 80/00

(52) U.S. Cl. CPC G16H 15/00; G16H 20/13; G16H 40/20; G16H 50/20; G16H 80/00

(57) ABSTRACT

A system for providing automated case management and reporting via artificial intelligence is provided. The system interacts with a user to obtain information from the user, which is then provided to an artificial intelligence engine to determine a medical complaint of the user. The artificial intelligence engine proceeds to automatically generate a digital record for the user that includes a predicted assessment and plan for treating the medical complaint. If standing order protocols exist, the digital records may be finalized and provided to the user, to a medical billing system, and to third parties for review and processing. Otherwise, the system may provide the digital record to a physician for further review to confirm the predictions and information associated with the digital record or to update the digital record. The digital record is then finalized and reported to the user, the medical billing system, and other third parties.

### CROSS REFERENCE TO RELATED APPLICATIONS

The present application claims priority to and the benefit of U.S. Provisional Patent Application No. 63/416,753, filed on Oct. 17, 2022, the entirety of which is hereby incorporated by reference in its entirety.

### FIELD OF THE INVENTION

The present application relates to artificial intelligence technologies, machine learning technologies, automation technologies, human and computer interaction technologies, sensor technologies, case management technologies, reporting technologies, healthcare onboarding technologies, diagnosis technologies, data analysis technologies, and, more particularly, to an artificial intelligence system and accompanying methods for providing automated case management and reporting, such as through conducting interactions between computing devices and users.

### BACKGROUND

In today's society, it has become increasingly important to be able to optimize patient onboarding, treatment, and reporting processes. Notably, patient onboarding and treatment often involves a manual trial and error process that typically differs from hospital to hospital or from medical practice to medical practice. While the provisioning of medicine to patients requires adherence to standards of care that are well-known in each medical specialty, the processes and policies patients need to follow before and after an appointment with a physician are much less standardized. For example, the trial and error process often results in repeated visits for the same medical condition, unnecessary visits to hospitals or medical practices, ineffective communications with patients, variations in the instructions or formats for recommended care, and ineffective patient follow-up procedures. Such a trial and error process often leaves patients exhausted, confused, and many times non-compliant with the care recommended by a physician to treat a medical condition of a patient. Additionally, the combination of clinical uncertainty and process uncertainty drives a constant loop of trial and error with patients. Still further, new patient demands or observations of existing conditions, new objective data compiled by physicians, and check-out errors, both clinical and administrative, all serve to drive a continual loop of often unnecessarily and wasteful work.

Currently, there are a variety of technologies, methodologies, and techniques utilized to process patients, coordinate physician appointments, and arrange for medical treatments. Such technologies, methodologies, and techniques include, but not limited to, software and hardware systems utilized to intake patient data, schedule appointments with physicians, store patient records, discharge patients, conduct medical billing, or a combination thereof. While existing technologies have been helpful in maintaining patient data and reducing certain administrative burdens on the medical system, such technologies often only optimize certain aspects of the patient onboarding and outboarding process, such as patient registration or medical billing. Additionally, existing systems often involve utilizing multiple solutions provided by multiple third parties that have software systems that to not readily integrate or communicate with solutions provided by other third parties, thereby leading to potential lack of interoperability and multiple points of failure in the medical system.

Based on at least the foregoing, there remains room for substantial enhancements to existing technologies and processes and for the development of new technologies and processes to facilitate patient onboarding and outboarding. For example, current technologies may be improved and enhanced so as to provide for more effective and uniform patient intake and registration, improved medical appointment scheduling, enhanced diagnostic capabilities, greater quality data, faster processing of patient data, improved medical record generation capabilities, and other enhancements. Such enhancements and improvements to methodologies and technologies may provide for increased standardization for patient onboarding and outboarding, improved patient compliance, reduced administrative work, reduce staff errors in order creation and routing, reduces the need for trial and error processes, among other benefits.

### SUMMARY

A system and accompanying methods for providing automated case management and reporting using artificial intelligence are disclosed. In particular, the system and methods provide a patient onboarding and outboarding platform incorporating algorithms that facilitate patient intake, generation of a physician-ready triage note, generation of a differential patient diagnosis, generation of a treatment plan and creation of orders prior to a physician examining a patient. In certain embodiments, the system and methods combine a view on probabilistic diagnosis and treatment suggestions available to the physician and patient alike, while also creating adjoining orders and instructions to the patient through discharge that are consistent with the billing instructions for the patient. As a result, a physician or the physician's staff may only need to verify, edit, or finalize the treatment plan and orders generated by utilizing the system and methods. Furthermore, in certain embodiments, as objective data is gathered from labs, physicians, imaging centers, or from patient subjective follow-up, the algorithms supporting the functionality of the system and methods may automatically re-run and update the digital records (e.g., notes or other records) for the patient to identify changes to the patient treatment plan and to identify possible new orders. In certain embodiments, the system and methods may also enable physicians and staff to review and edit the updated records to confirm accuracy.

In certain embodiments, a system for providing automated case management and reporting using artificial intelligence is provided. The system may include a memory that stores instructions and a processor that executes the instructions to perform various operations of the system. The system may perform an operation that includes registering, such as via an interface of the system, an individual with the system. For example, the registering may include, but is not limited to, obtaining demographic information, identification information, psychographic information, insurance information, payment information, any type of information, or a combination thereof. Additionally, in certain embodiments, the registration may include obtaining consents from the individual to perform medical procedures, consents to be examined, consents to perform any other type of activity that may be consented to, or a combination thereof. The system may then perform an operation that includes interacting with the individual to obtain information from the individual, such as information relating to a medical condition or complaint. In certain embodiments, the interacting may be conducted by utilizing a triage artificial intelligence engine. The system may perform an operation that includes identifying, by utilizing the triage artificial intelligence engine and based on the information, the medical complaint associated with the individual. The medical complaint may be identified based on the information having a correlation with medical complaint information that is utilized to train the triage artificial intelligence engine.

The system may proceed to perform an operation that includes generating, by utilizing the triage artificial intelligence engine and based on the medical complaint, a digital record associated with the individual. In certain embodiments, the digital record may include a plan associated with addressing the medical complaint. In certain embodiments, the digital record may be or may include a Subjective, Objective, Assessment, and Plan (S.O.A.P.) note. In certain embodiments, if a standing order protocol exists that matches criteria associated with the plan in the digital record, the digital record for the patient may be finalized by the system. If, however, the standing order protocol does not exist, the system may perform an operation that includes providing the digital record for further review to a provider. The provider may review the digital record and provide input. The system may receive the input and may modify the digital record based on the input. The digital record may then be utilized for billing, ordering and scheduling treatments and procedures, obtaining medicine, performing a variety of other actions, or a combination thereof.

In certain embodiments, a method for automated case management and reporting using artificial intelligence is disclosed. The method may include a memory that stores instructions and a processor that executes the instructions to perform the functionality of the method. In particular, the method may include interacting, such as by utilizing a triage artificial intelligence engine of a system, with an individual to obtain information from the individual during an encounter with the individual. Additionally, the method may include identifying, by utilizing the triage artificial intelligence engine and based on the information, a medical complaint associated with the individual. In certain embodiments, the medical complaint may be identified based on the information having a correlation with medical complaint information utilized to train the triage artificial intelligence engine. For example, the information may have a threshold amount of correlation with information used to train the triage artificial intelligence engine to detect or determine medical complaints. The method may also include generating, by utilizing the triage artificial intelligence engine and based on the medical complaint, a digital record associated with the individual. In certain embodiments, the digital record may include a plan associated with addressing the medical complaint. If a standing order protocol exists that matches criteria associated with the plan in the digital record, the method may include finalizing (e.g., completing) the digital record. If, however, the standing order protocol does not exist, the method may include providing the digital record for further review by a provider. Moreover, the method may include modifying the digital record based on an input received that is associated with the further review.

According to further embodiments, a computer-readable device comprising instructions, which, when loaded and executed by a processor cause the processor to perform operations, the operations comprising: interacting, by utilizing a triage artificial intelligence engine of a system, with an individual to obtain information from the individual during a first encounter with the individual; identifying, by utilizing the triage artificial intelligence engine and based on the information, a medical complaint associated with the individual, wherein the medical complaint is identified based on the information having a correlation with medical complaint information utilized to train the triage artificial intelligence engine; generating, by utilizing the triage artificial intelligence engine and based on the medical complaint, a digital record associated with the individual, wherein the digital record includes a plan associated with addressing the medical complaint; finalizing the digital record if a standing order protocol exists that matches criteria associated with the plan in the digital record; providing, if the standing order protocol does not exist, the digital record for further review by a provider; and modifying, based on an input received that is associated with the further review, the digital record.

These and other features of the systems and methods for automated case management and reporting using artificial intelligence are described in the following detailed description, drawings, and appended claims.

### BRIEF DESCRIPTION OF THE DRAWINGS

FIG. 1 is a schematic diagram of a system for providing automated case management and reporting according to embodiments of the present disclosure.

FIG. 2 illustrates an exemplary process flow for use with the system of FIG. 1 that enables patient registration, generates digital records including plans for the patient, facilitates patient encounters with a provider, and facilitates digital record editing according to embodiments of the present disclosure.

FIG. 3 illustrates an exemplary process flow for use with the system of FIG. 1 that facilitates updates to digital records of a patient, validates plans for patients, and facilitates medical billing according to embodiments of the present disclosure.

FIG. 4 is a flow diagram illustrating a sample method for providing automated case management and reporting using artificial intelligence according to embodiments of the present disclosure.

FIG. 5 is a flow diagram illustrating a sample method for providing an artificial intelligence system including an inductive reasoning engine for enabling users to request specific plans, treatments, and/or medications according to embodiments of the present disclosure.

FIG. 6 is a schematic diagram of a machine in the form of a computer system within which a set of instructions, when executed, may cause the machine to facilitate the automation of case management and reporting according to embodiments of the present disclosure.

### DETAILED DESCRIPTION OF THE DRAWINGS

A system 100 and accompanying methods for providing automated case management and reporting using artificial intelligence are disclosed. In particular, the system 100 and methods provide a patient onboarding and outboarding platform incorporating algorithms that facilitate patient intake, generation of a physician-ready triage note, generation of a differential patient diagnosis, generation of a treatment plan, and creation of orders prior to a physician examining a patient. In certain embodiments, the system 100 and methods combine a view on probabilistic diagnosis and treatment suggestions available to the physician and patient, while also creating orders and instructions to the patient through discharge that are consistent with the billing instructions for the patient. Based on the functionality provided by the system 100 and methods, a physician or the physician's staff may only need to verify, edit, or finalize the treatment plan and orders generated by utilizing the system 100 and methods. In certain embodiments, as objective data is gathered from labs, physicians, imaging centers, or from patient subjective follow-up, the algorithms supporting the functionality of the system 100 and methods may automatically re-run and update the digital records (e.g., notes or other records) for the patient to identify changes to the patient treatment plan and to identify possible new orders. In certain embodiments, the system and methods may also enable physicians and staff to review and edit the updated records to confirm accuracy prior to being finalized.

In accordance with embodiments of the present disclosure, the system 100 and methods may include a unique and integrated clinical and administrative onboarding and outboarding software platform. To support the functionality of the onboarding and outboarding software platform, the system 100 and methods may utilize one or more artificial intelligence models, which may be trained using data to facilitate performance of a plurality of operations of the platform. For example, in certain embodiments, the system 100 and methods may include a triage artificial intelligence engine that may utilize evidence-based research libraries to facilitate generation of triage questions for patient intake, propose high-probability assessment codes (e.g., diagnosis codes), generate recommendations and predictions for treatment plans for medical complaints of patients, and perform a variety of other operative functionality of the system. As another example, in certain embodiments, the system 100 and methods may include a physician assessment engine that may serve as a decision engine that processes collected data to generate recommendations and workflow direction. In certain embodiments, the physician assessment engine may include standing order protocols, which can generate automatic processes for completion. In certain embodiments, standing order protocols may comprise decision trees defined with parameters that may be configured to execute specific actions.

In certain embodiments, the system 100 and methods may include having a user (e.g., a patient) connect with the onboarding and outboarding platform, such as via a device of the user. For example, an application supporting the functionality of the system 100 and methods may execute on the device and may provide an interface on the device that is configured interact with and receive inputs from the patient. In certain embodiments, as a preliminary matter, the platform may register with the system 100. Questions and intake forms may be presented to the user, such as via the interface. The user may input any type of information into the interface for the purposes of the registration with the system 100. Such information may include, but is not limited to, demographic information, identity information, insurance information, psychographic information, location information, contact information, any type of information, or a combination thereof. In certain embodiments, the registration process may also include providing consents to the user via the user interface. The consents may be utilized to obtain consent from the user to perform examinations, medical procedures, treatments, or a combination thereof. In certain embodiments, payment information, biometric information, login credentials, and any other information may also be input during the registration process.

Once the user is registered with the system 100, the system 100 and methods may include asking a series of questions to the user, such as by utilizing the triage artificial intelligence engine, to determine the user's primary medical complaint. In certain embodiments, the questions may be posed using text, voice, image content, video content, any other method, or a combination thereof. The questions may relate to the medical complaint that the user is experiencing and may be utilized to gather any relevant triage information. The responses to the questions may be provided by the user using text, voice, image content, video content, any other method, or a combination thereof. In certain embodiments, the triage artificial intelligence engine may be configured to create an in-depth review of the user's responses to collect a basic pre-consultation triage similar to what a medical staff member would complete in a physician's office. In certain embodiments, the triage artificial intelligence engine may also utilize the information obtained from the user to determine the chief medical complaint, such as by utilizing the functionality provided by an artificial intelligence model of the triage artificial intelligence engine.

The information obtained from the user and the review of the information may be included in a digital record (e.g., a digital note) serving as the clinical documentation for the user. In certain embodiments, the triage artificial intelligence engine may also generate predictions relating to the most likely assessment codes, which may be utilized for billing purposes, treatment purposes, and information purposes. The predicted assessment codes may also be incorporated into the digital record of the user. Depending on the generated assessment, the system 100 and methods may generate a proposed treatment plan, such as based on evidence-based clinical reasoning.

The information collected from the user and the information generated by the triage artificial intelligence engine may then be provided to the physician assessment engine for further processing, analysis, or a combination thereof. In certain embodiments, the physician assessment engine may facilitate a plurality of actions based various conditions. For example, in an exemplary scenario, if there are existing standing order protocols (i.e., decision trees defined with narrow parameters that can execute specific action) in place that match criteria based on the user's inputted information and responses to the questions posed by the system 100, the system 100 may generate or obtain corresponding orders, education resources, or a combination thereof, to the user without further input from the user. Additionally, in certain embodiments, the digital record may be updated to include information associated with the orders, education resources, or a combination thereof. Furthermore, in certain embodiments, the orders, education resources, or a combination thereof, may be provided to the user, such as via a transmission to a user device of the user. The system and methods may then close out the encounter with the user, digitally mark the digital record as completed, and then transfer the digital record to a billing system for medical billing purposes.

If, however, further review of the digital record and/or proposed treatment plan is required and the assessment generated by the triage artificial intelligence engine does not require that the user have a face-to-face consultation with a physician, the digital record may be included in a worklist for the physician to review and address, such as asynchronously. The physician may review the information contained in the digital record, along with any other case-related information associated with the user, and provide an assessment, orders, referrals, patient education information, or a combination thereof. In certain embodiments, if the physician (or others) need additional information from the user, a messaging utility of the system may facilitate direct communication with the user. For example, a chat or messaging feature may be included in the system that initiates a digital messaging session with the user to request additional information from the user. The user may then input the requested information, such as via a user interface of a user device of the user and transmit the requested information to the system. Based on the received information, the digital record may be updated, such as by the physician and/or the system, and the digital record may be marked as complete. The encounter may then be closed out with the user and the digital record may then be transferred to a billing system for medical billing purposes.

On the other hand, if further review is required and the assessment generated by the triage artificial intelligence engine requires a face-to-face consultation of the user with the physician, a telemedicine visit may be required. In certain embodiments, if there are available resources at the present time, the option may be presented to the user to enter a waiting room, such as a digital waiting room on an application executable on a user device of the user. In certain embodiments, such as if resources are not currently available, a notification may be transmitted to the user that indicates that they will be contacted by a physician. The user's digital record may then be passed to a customer service representative worklist for review and scheduling. In certain embodiments, the user may opt to select an in-person visit instead of a telemedicine visit. In such a scenario, the system and methods may include determining a plurality of potential facilities within a certain vicinity of the user that the user may visit for an in-person visit with a physician. The user may then schedule, such as via a user device, an in-person visit at a facility, such as a hospital. In certain embodiments, for example, the system and methods may incorporate the use of an integrated video chat within the platform that will enable the physician to connect to the user for a face-to-face consultation. Using the information gathered, the physician may then complete the digital record (e.g., the S.O.A.P. note) by adding an additional information, assessments, plans, orders, referrals, patient education, or a combination thereof. Once the digital record is completed, the encounter with the user may be closed out and the digital record may then be transferred to a billing system for medical billing purposes.

In certain embodiments and as another possible scenario, depending on the assessment generated by the triage artificial intelligence engine, the system and methods may include automatically generating a “fall-out” and presenting the user with a message information the user to proceed to an emergency room immediately. For example, in certain embodiments of such a scenario, no digital record (i.e., the S.O.A.P note) may be generated by the system, however, contact and other information for the user may be retained and placed in a worklist for follow-up by the customer service representative associated with the system.

In certain embodiments, when the digital record is market complete under some or all of the above-described scenarios, assessment and/or diagnoses codes may be transferring to the medical billing system for medical billing purposes. In certain embodiments, any orders that need to be processed by third parties may be transferred via third party setup process. In certain embodiments, for example, orders and/or codes may be transferred via fax, secure messages, integrated solutions, digital transmissions, or a combination thereof.

After an initial encounter with the user, post encounter medical or other results, such as those provided by connected third parties or from at-home test result collection kits may be added to the digital record. The system and methods may include utilizing a standard of care reference library to facilitate conversion of lab values to low, normal, high or other scale. The lab values may then be based pack through to the triage artificial intelligence engine for review. In certain embodiments, the triage artificial intelligence engine may utilize artificial intelligence model to facilitate the comparison of the lab results against information contained in the reference library and may return potential new assessments and/or treatment plan recommendations for the user. In certain embodiments, lab values, new assessments, treatment plan updates, or a combination thereof, may be provided to the physician assessment engine. In certain embodiments, if a standard order protocol(s) exist, the system and methods may include executing the prescribed action(s), if any. If, however, a standard order protocol does not exist, the system may utilize the updated digital record may be passed back to the physician review worklist. Once the updated digital record is reviewed by the physician, results and any new orders, recommendations, or both, may be sent to the user via the system, such as via a client portal of the system. In certain embodiments, all closed and/or completed digital records associated with the user may be stored in the system based on guidelines (e.g., HIPAA guidelines) for future use.

If a user returns for a new encounter with the system, such as if the user is being seen by a physician for a follow-up for a procedure performed on the patient, the previous information associated with the user may be copied and presented to the user. The user may be asked, such as via a user interface, to review if any of the information in the digital record has changed. If there are any changes, the updated information provided by the user may be provided to the triage artificial intelligence system for further processing and analysis. If there are no changes to be made, the digital record may enter the physician assessment engine and continue via the processes described above. If the user is being seen for a new or unrelated encounter, the new information for the new encounter will be provided to the triage artificial intelligence engine and a new digital record for the encounter may be generated and processed using the processes described herein. In certain embodiments, all completed digital records, orders, referrals, patient education materials, and/or any other information may be made available to the patient for review and/or downloaded using the user's login credentials submitted via a client portal of the system. The credentials may be generated upon completion of a new patient information collection process, such as when the user first registers with the system.

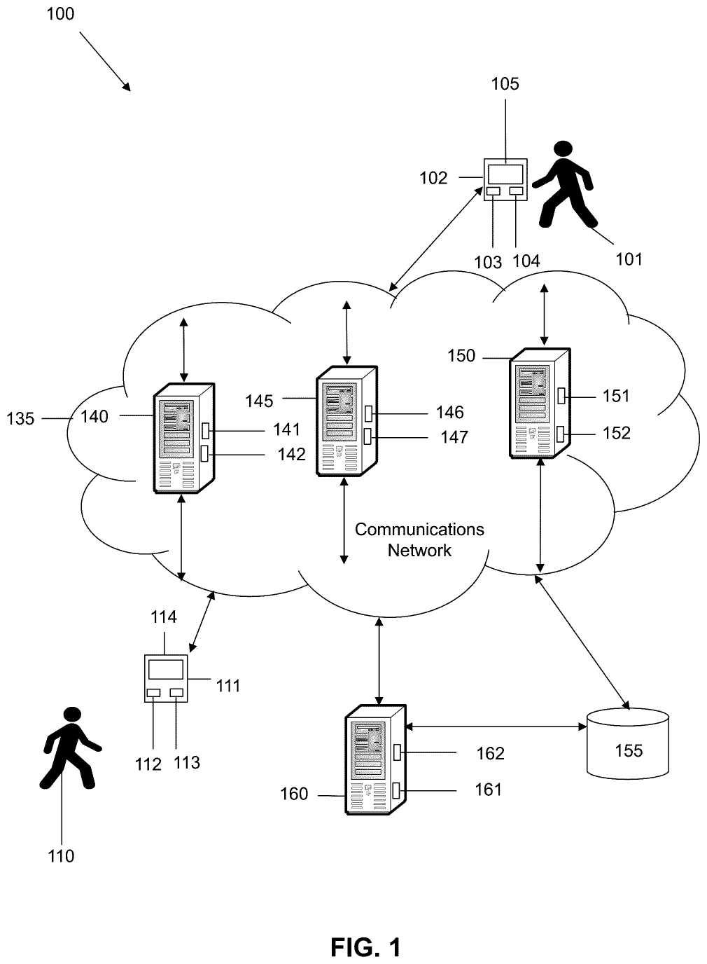

As shown in FIG. 1, a system for providing automated case management and reporting according to embodiments of the present disclosure is disclosed. Notably, the system 100 may be configured to support, but is not limited to supporting, healthcare systems, patient intake systems, patient digital records systems, medical diagnosis systems, automation systems, data analytics systems and services, data collation and processing systems and services, artificial intelligence services and systems, machine learning services and systems, content delivery services, cloud computing services, satellite services, telephone services, voice-over-internet protocol services (VoIP), software as a service (SaaS) applications, platform as a service (PaaS) applications, social media applications and services, operations management applications and services, productivity applications and services, mobile applications and services, and/or any other computing applications and services. Notably, the system 100 may include a first user 101, who may utilize a first user device 102 to access data, content, and services, or to perform a variety of other tasks and functions. As an example, the first user 101 may utilize first user device 102 to transmit signals to access various online services and content, such as those available on an internet, on other devices, and/or on various computing systems. As another example, the first user device 102 may be utilized to access an application, devices, and/or components of the system 100 that provide any or all of the operative functions of the system 100. For example, the first user 101 may utilize the first user device 102 to access an application having a user interface that enables the first user 101 to submit personal data into the system 100 to register the first user 101 with the system 100 for purposes of patient intake and examination by a provider (e.g., second user 110 or a hospital system at which the second user 110 works at). In certain embodiments, the first user 101 may be a bystander, any type of person, a robot, a humanoid, a program, a computer, any type of user, or a combination thereof, that may be located in a particular environment.

In certain embodiments, the first user 101 may be a person that may be experiencing a medical condition, may be seeking to having a health checkup, may be seeking a medical treatment, or a combination thereof. For example, the first user 101 may be a patient of a physician (e.g., the second user 110). In certain embodiments, the first user device 102 may be utilized by the first user to interact with the system 100, other users of the system 100, or a combination thereof. In certain embodiments, the first user device 102 may include a memory 103 that includes instructions, and a processor 104 that executes the instructions from the memory 103 to perform the various operations that are performed by the first user device 102. In certain embodiments, the processor 104 may be hardware, software, or a combination thereof. The first user device 102 may also include an interface 105 (e.g. screen, monitor, graphical user interface, etc.) that may enable the first user 101 to interact with various applications executing on the first user device 102 and to interact with the system 100. In certain embodiments, the first user device 102 may be and/or may include a computer, any type of sensor, a laptop, a set-top-box, a tablet device, a phablet, a server, a mobile device, a smartphone, a smart watch, and/or any other type of computing device. Illustratively, the first user device 102 is shown as a smartphone device in FIG. 1. In certain embodiments, the first user device 102 may be utilized by the first user 101 to control and/or provide some or all of the operative functionality of the system 100.

In addition to using first user device 102, the first user 101 may also utilize and/or have access to additional user devices. As with first user device 102, the first user 101 may utilize the additional user devices to transmit signals to access various online services and content. The additional user devices may include memories that include instructions, and processors that executes the instructions from the memories to perform the various operations that are performed by the additional user devices. In certain embodiments, the processors of the additional user devices may be hardware, software, or a combination thereof. The additional user devices may also include interfaces that may enable the first user 101 to interact with various applications executing on the additional user devices and to interact with the system 100. In certain embodiments, the first user device 102 and/or the additional user devices may be and/or may include a computer, any type of sensor, a laptop, a set-top-box, a tablet device, a phablet, a server, a mobile device, a smartphone, a smart watch, and/or any other type of computing device, and/or any combination thereof. Sensors may include, but are not limited to, cameras, motion sensors, acoustic/audio sensors, pressure sensors, temperature sensors, light sensors, heart-rate sensors, blood pressure sensors, sweat detection sensors, breath-detection sensors, stress-detection sensors, any type of health sensor, humidity sensors, any type of sensors, or a combination thereof.

The first user device 102 and/or additional user devices may belong to and/or form a communications network. In certain embodiments, the communications network may be a local, mesh, or other network that enables and/or facilitates various aspects of the functionality of the system 100. In certain embodiments, the communications network may be formed between the first user device 102 and additional user devices through the use of any type of wireless or other protocol and/or technology. For example, user devices may communicate with one another in the communications network by utilizing any protocol and/or wireless technology, satellite, fiber, or any combination thereof. Notably, the communications network may be configured to communicatively link with and/or communicate with any other network of the system 100 and/or outside the system 100.

In certain embodiments, the first user device 102 and additional user devices belonging to the communications network may share and exchange data with each other via the communications network. For example, the user devices may share information associated with a user (e.g., patient) with each other, information associated with digital records generated and/or maintained by the system 100, information relating to lab results, information relating to medical or physical examinations conducted by a physician on a user, information relating to the various components of the user devices, information associated with images and/or content accessed by a user of the user devices, information identifying the locations of the user devices, information indicating the types of sensors that are contained in and/or on the user devices, information identifying the applications being utilized on the user devices, information identifying how the user devices are being utilized by a user, information identifying user profiles for users of the user devices, information identifying device profiles for the user devices, information identifying the number of devices in the communications network, information identifying devices being added to or removed from the communications network, any other information, or any combination thereof.

In addition to the first user 101, the system 100 may also include a second user 110. The second user 110 may be a person that may facilitate treatment of the first user 101. For example, in certain embodiments, the second user 110 may be a physician, nurse, technician, intake professional, pharmacist, or other individual that work at a hospital, medical practice, any other location, or a combination thereof. In certain embodiments, the second user device 111 may be utilized by the second user 110 to transmit signals to request various types of content, services, and data provided by and/or accessible by communications network 135 or any other network in the system 100. In certain embodiments, the second user device 111 may be utilized by the second user 110 to view patient data, generate plans for patients, edit plants for patients, provide instructions for patients, confirm the content of digital records generated by the system 100, perform any operative functionality of the system 100, or a combination thereof. In further embodiments, the second user 110 may be a robot, a computer, a vehicle (e.g. semi or fully-automated vehicle), a humanoid, an animal, any type of user, or any combination thereof. The second user device 111 may include a memory 112 that includes instructions, and a processor 113 that executes the instructions from the memory 112 to perform the various operations that are performed by the second user device 111. In certain embodiments, the processor 113 may be hardware, software, or a combination thereof. The second user device 111 may also include an interface 114 (e.g. screen, monitor, graphical user interface, etc.) that may enable the first user 101 to interact with various applications executing on the second user device 111 and, in certain embodiments, to interact with the system 100. In certain embodiments, the second user device 111 may be a computer, a laptop, a set-top-box, a tablet device, a phablet, a server, a mobile device, a smartphone, a smart watch, and/or any other type of computing device. Illustratively, the second user device 111 is shown as a mobile device in FIG. 1. In certain embodiments, the second user device 111 may also include sensors, such as, but are not limited to, cameras, audio sensors, motion sensors, pressure sensors, temperature sensors, light sensors, heart-rate sensors, blood pressure sensors, sweat detection sensors, breath-detection sensors, stress-detection sensors, any type of health sensor, humidity sensors, any type of sensors, or a combination thereof.

In certain embodiments, the first user device 102, the additional user devices, and/or the second user device 111 may have any number of software applications and/or application services stored and/or accessible thereon. For example, the first user device 102, the additional user devices, and/or the second user device 111 may include applications for controlling and/or accessing the operative features and functionality of the system 100, applications for controlling and/or accessing any device of the system 100, healthcare applications, patient record management applications, patient record generating applications, medical billing applications, interactive social media applications, biometric applications, cloud-based applications, VoIP applications, other types of phone-based applications, product-ordering applications, business applications, e-commerce applications, media streaming applications, content-based applications, media-editing applications, database applications, gaming applications, internet-based applications, browser applications, mobile applications, service-based applications, productivity applications, video applications, music applications, social media applications, any other type of applications, any types of application services, or a combination thereof. In certain embodiments, the software applications may support the functionality provided by the system 100 and methods described in the present disclosure. In certain embodiments, the software applications and services may include one or more graphical user interfaces so as to enable the first and/or potentially second users 101, 110 to readily interact with the software applications. The software applications and services may also be utilized by the first and/or potentially second users 101, 110 to interact with any device in the system 100, any network in the system 100, or any combination thereof. In certain embodiments, the first user device 102, the additional user devices, and/or potentially the second user device 111 may include associated telephone numbers, device identities, or any other identifiers to uniquely identify the first user device 102, the additional user devices, and/or the second user device 111.

The system 100 may also include a communications network 135. The communications network 135 may be under the control of a service provider, any designated user, a computer, another network, or a combination thereof. The communications network 135 of the system 100 may be configured to link each of the devices in the system 100 to one another. For example, the communications network 135 may be utilized by the first user device 102 to connect with other devices within or outside communications network 135. Additionally, the communications network 135 may be configured to transmit, generate, and receive any information and data traversing the system 100. In certain embodiments, the communications network 135 may include any number of servers, databases, or other componentry. The communications network 135 may also include and be connected to a mesh network, a local network, a cloud-computing network, an IMS network, a VoIP network, a security network, a VoLTE network, a wireless network, an Ethernet network, a satellite network, a broadband network, a cellular network, a private network, a cable network, the Internet, an internet protocol network, MPLS network, a content distribution network, any network, or any combination thereof. Illustratively, servers 140, 145, and 150 are shown as being included within communications network 135. In certain embodiments, the communications network 135 may be part of a single autonomous system that is located in a particular geographic region or be part of multiple autonomous systems that span several geographic regions.

Notably, the functionality of the system 100 may be supported and executed by using any combination of the servers 140, 145, 150, and 160. The servers 140, 145, and 150 may reside in communications network 135, however, in certain embodiments, the servers 140, 145, 150 may reside outside communications network 135. The servers 140, 145, and 150 may provide and serve as a server service that performs the various operations and functions provided by the system 100. In certain embodiments, the server 140 may include a memory 141 that includes instructions, and a processor 142 that executes the instructions from the memory 141 to perform various operations that are performed by the server 140. The processor 142 may be hardware, software, or a combination thereof. Similarly, the server 145 may include a memory 146 that includes instructions, and a processor 147 that executes the instructions from the memory 146 to perform the various operations that are performed by the server 145. Furthermore, the server 150 may include a memory 151 that includes instructions, and a processor 152 that executes the instructions from the memory 151 to perform the various operations that are performed by the server 150. In certain embodiments, the servers 140, 145, 150, and 160 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 140, 145, 150 may be communicatively linked to the communications network 135, any network, any device in the system 100, or any combination thereof.

The database 155 of the system 100 may be utilized to store and relay information that traverses the system 100, cache content that traverses the system 100, store data about each of the devices in the system 100 and perform any other typical functions of a database. In certain embodiments, the database 155 may be connected to or reside within the communications network 135, any other network, or a combination thereof. In certain embodiments, the database 155 may serve as a central repository for any information associated with any of the devices and information associated with the system 100. Furthermore, the database 155 may include a processor and memory or may be connected to a processor and memory to perform the various operation associated with the database 155. In certain embodiments, the database 155 may be connected to the servers 140, 145, 150, 160, the first user device 102, the second user device 111, the additional user devices, any devices in the system 100, any process of the system 100, any program of the system 100, any other device, any network, or any combination thereof.

The database 155 may also store information and metadata obtained from the system 100, store metadata and other information associated with the first and second users 101, 110, store artificial intelligence models utilized in the system 100, store sensor data and/or content obtained from a patient, store predictions made by the system 100 and/or artificial intelligence models, storing confidence scores relating to predictions made, store threshold values for confidence scores, responses outputted and/or facilitated by the system 100, store information associated with anything determined or detected via the system 100, store information and/or content utilized to train the artificial intelligence models, store information associated with behaviors and/or actions conducted by individuals, store user profiles associated with the first and second users 101, 110, store device profiles associated with any device in the system 100, store communications traversing the system 100, store user preferences, store information associated with any device or signal in the system 100, store information relating to patterns of usage relating to the user devices 102, 111, store any information obtained from any of the networks in the system 100, store historical data associated with the first and second users 101, 110, store device characteristics, store information relating to any devices associated with the first and second users 101, 110, store information associated with the communications network 135, store any information generated and/or processed by the system 100, store any of the information disclosed for any of the operations and functions disclosed for the system 100 herewith, store any information traversing the system 100, or any combination thereof. In certain embodiments, the database 155 may be configured to store information supplied by the patient to register with the system 100, information associated with the patient's health status, digital records, lab results, information associated with surgical procedures to be performed or already performed on the patient, plans generated by the system 100, edits to plans generated by the system 100, medical billing information, insurance information, information relating to patient visits and medical conditions, information associated with medical complaints made by a patient or determined by the system 100, information associated with recommendations for treatments to be done for the patient, information associated with medication to be taken by the patient, information identifying standing order protocols, information identifying the patient and/or physician, any other information of the system 100, or a combination thereof. Furthermore, the database 155 may be configured to process queries sent to it by any device in the system 100.

In certain embodiments, the system 100 may incorporate the use of any number of artificial intelligence engines, such as, but not limited to, a triage artificial intelligence engine 204 and a physician assessment engine 208. In certain embodiments, the system 100 may include one or more artificial intelligence models supporting the functionality of the system 100, a triage artificial intelligence engine 204 and the physician assessment engine 208. In certain embodiments, an artificial intelligence model may be a file, program, module, and/or process that may be trained by the system 100 (or other system) to recognize certain patterns, diagnoses, health conditions, diseases, behaviors, and/or content. For example, the artificial intelligence model(s) may be trained to determine medical complaints (i.e., what the user is currently experiencing from a health standpoint), detect specific types of diseases afflicting a user of the system 100, generate a plan to treat detection diseases and/or conditions, generate assessment codes (e.g., CPT codes or other codes) that may be utilized for billing purposes or for obtaining prescriptions. In certain embodiments, the artificial intelligence model may be, may include, and/or may utilize a Deep Convolutional Neural Network, a one-dimensional convolutional neural network, a two-dimensional convolutional neural network, a Long Short-Term Memory network, any type of machine learning system, any type of artificial intelligence system, or a combination thereof. Additionally, in certain embodiments, the artificial intelligence model may incorporate the use of any type of artificial intelligence and/or machine learning algorithms to facilitate the operation of the artificial intelligence model(s).

The system 100 may train the artificial intelligence model(s) to reason and learn from data fed into the system 100 so that the model(s) may generate and/or facilitate the generation of predictions about new data and information that is fed into the system 100 for analysis. For example, the system 100 may train an artificial intelligence model using various types of data, information, and/or content, such as, but not limited to, images, video content, audio content, text content, augmented reality content, virtual reality content, information relating to patterns, information relating to behaviors, information relating to characteristics of users, information relating to environments, sensor data, information from medical libraries, information associated with diseases or medical conditions, any data associated with the foregoing, any type of data, or a combination thereof. In certain embodiments, the content and/or data utilized to train the artificial intelligence model may be utilized to correlate and/or associate user-provided information to specific detectable medical conditions, medical treatment plans, assessments, and the like. As additional data and/or content is fed into the model(s) over time, the model's ability to recognize medical complaints, generate plans, and determine assessments will improve and be more finely tuned.

In certain embodiments, the triage artificial intelligence engine may be configured to engage and/or interact with a user to facilitate identification of a medical complaint associated with the user. For example, the triage artificial intelligence engine may be configured to transmit messages to the user via an application accessible by the first user device 102 requesting that the user provide information associated with what the user is feeling, what symptoms the user has, any other information, or a combination thereof. Based on the interactions with the user, the triage artificial intelligence engine may determine the chief medical complaint of the user (e.g., what the user is complaining about from a health standpoint). Additionally, in certain embodiments, the triage artificial intelligence engine may be configured to facilitate the automatic generation of digital record that may include a ready-for-execution medical note, such as a S.O.A.P. note. The note, for example, may include a predicted assessment (e.g., diagnosis or medical condition) for the user, a treatment plan for treating the diagnosis or medical condition, subject information associated with the user, and objective data associated with the user (e.g., lab results, measurements, vital signs, etc.). In certain embodiments, assessment codes (CPT codes or other codes that may be used for billing purposes or insurance purposes) may also be predicted by the triage artificial intelligence system.

In certain embodiments, the physician assessment engine, may be configured to facilitate confirmation of the information in the auto-generated digital record, such as by providing the digital record to a physician for further review. In certain embodiments, the physician assessment engine may also be configured to analyze the digital record and/or information associated with the user to determine whether to provide the digital record to a physician for further review or to finalize the digital record directly, such as if standing order protocols associated with the medical complaint and/or predicted assessment for the user exist. In certain embodiments, the physician assessment engine may also be utilized to determine whether the user needs a face-to-face encounter with the physician, whether an in-person encounter is required, whether testing is to be performed on the user, whether a procedure is to be performed with respect to the user, whether the user should go directly to a hospital or other facility within a vicinity of the location of the user, along with other functional described in the present disclosure. The physician assessment engine may be configured to perform its operative functionality by utilizing any number of artificial intelligence models and functionality.

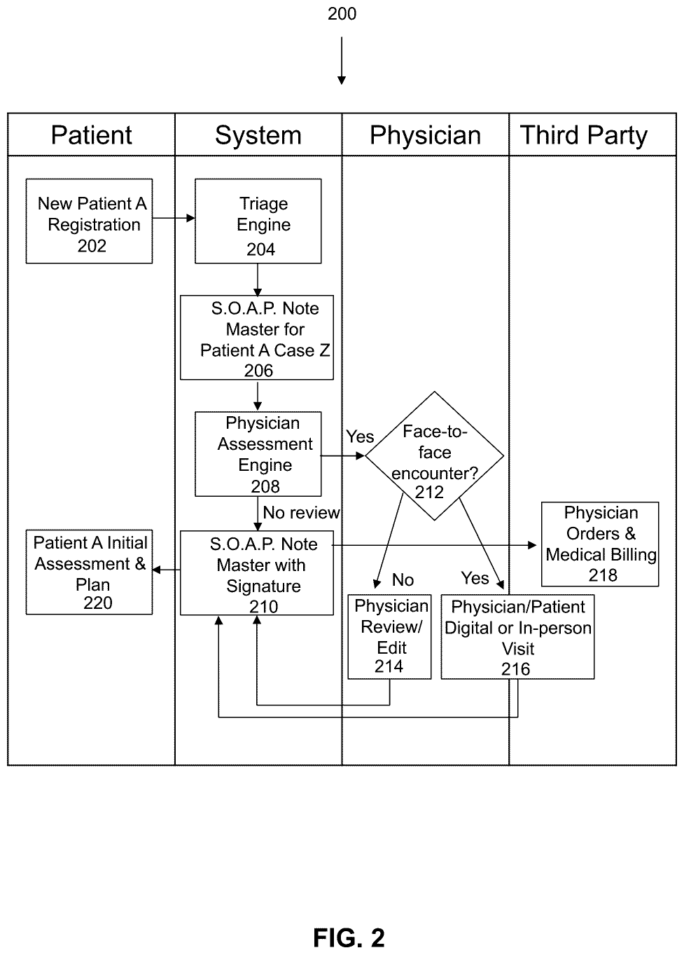

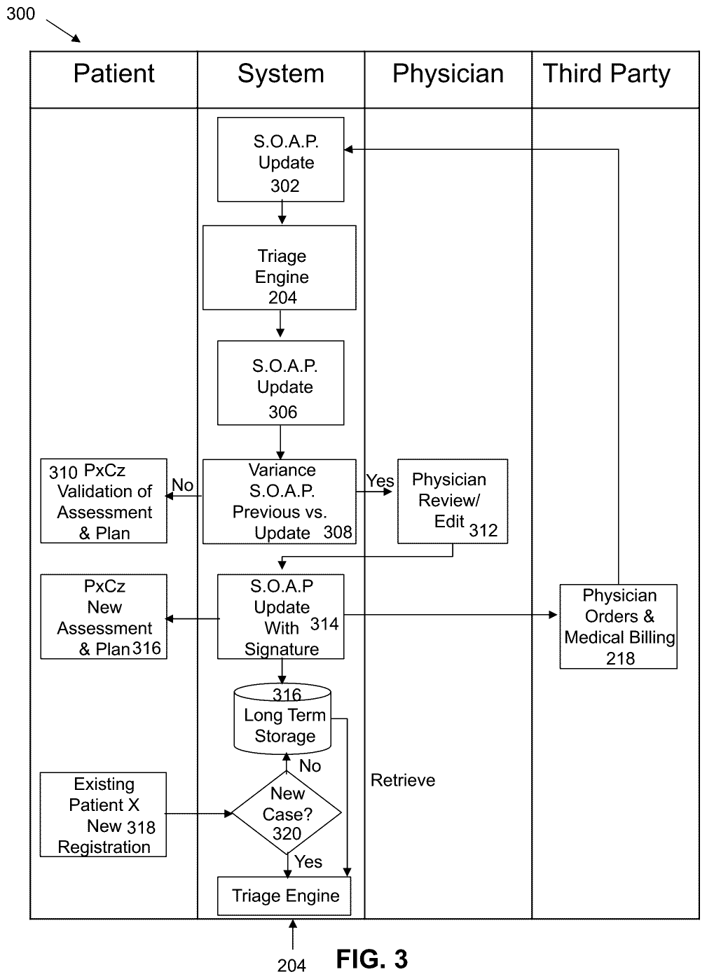

Operatively, the system 100 may operate and/or execute the functionality as described and illustrated in FIGS. 2 and 3 or as otherwise described herein. FIG. 2 illustrates an exemplary process flow for use with the system 100 that enables patient registration, generates digital records including plans for the patient, facilitates patient encounters with a provider, and facilitates digital record editing according to embodiments of the present disclosure. FIG. 3 illustrates an exemplary process flow for use with the system 100 that facilitates updates to digital records of a patient, validates plans for patients, and facilitates medical billing according to embodiments of the present disclosure. Referring initially to FIG. 2, the process flow 200 may include, at 202, registering a patient with the system 100. For example, the user (e.g., first user 101) may access an application supporting the functionality of the system 100 by utilizing first user device 102. The application functionality and features may be accessible by rendered graphical user interface that may be displayed on an interface of the first user device 102. The user may register may inputting demographic information, psychographic information, identity information, location information, physiological information, any other information, or a combination thereof. In certain embodiments, during the registration process, the user may also be provided with consent forms that may required the user's consent or authorization before the user may have an examination, procedure, or treatment.

At 204, the flow 200 may include utilizing the triage artificial intelligence engine 204 to interact with the user to extra further information from the user to determine the user's medical complaint. For example, the triage artificial intelligence engine 204 may pose questions to the user, the responses to which may be utilized to determine the medical complaint. Such questions may include questions relating to the symptoms that the user is experiencing, a history of such symptoms, the foods that the user ate, the medications that the user is taking, whether others in the user's vicinity are experiencing symptoms, any other questions, or a combination thereof. In certain embodiments, the triage artificial intelligence engine 204 may be configured to interact with the user via voice-based communications, text-based communications, video-related communications, augmented reality based communications, virtual reality based communications, any other communication technology, or a combination thereof. Once the user provides the information in response to the interactions with the engine, the process flow 200 may proceed to 206. At 206, the flow 200 may include auto-generating a digital record for the user. As indicated here, the digital record may include a digital S.O.A.P. note including subjective data associated with the user, objective data associated with the user, an assessment (e.g., diagnosis) for the user, a plan for treatment of the condition associated with the diagnosis, or a combination thereof. In certain embodiments, the note may include all the elements to treat and bill the user (e.g., Patient A, Case Z (“PaCz”)). In certain embodiments, the plan generated by the engine 204 may include information relating to labs, imaging, medication, medical equipment, and specialist treatment needed for the user.

The process flow 200 may include providing the digital record to the physician assessment engine 208 for review and processing. In certain embodiments, the physician assessment engine 208 may be configured to filter the digital record for the user to establish workflow priorities. For example, the physician assessment engine 208 may be configured to (1) determine whether the digital record is to be completed and signed as-is; (2) determining whether the physician needs to visually review the digital record prior to completion and signing; (3) whether the user requires a telemedicine or in-person visit with the physician; and (4) whether the user needs to go to the hospital or other treatment facility immediately. In certain embodiments, while generic standards of care may be embedded in the triage artificial intelligence engine 204, rendering provider/physician specific protocols to create proper workflow priority may be required. For example, the physician assessment engine 208 may determine that the digital record does not need further review because standing order protocols that dictate the protocol for the specific diagnosis for the user already exist. In such a scenario, the flow 200 may proceed to 210 where the digital record may be finalized and marked complete by the system 100. The digital record (or at least the digital S.O.A.P. note) may be signed by the physician (e.g. via digital signature, authentication (e.g., biometric), or other technique). In certain embodiments, the finalized digital record may include all elements to treat and bill the user. The plan of the digital record may identify labs, imaging, medications, medical equipment, and specialist treatment needed for the user. The digital record may be utilized to inform and educate the user as well as provide instruction to third parties on the method of care needed for the user. The digital record may be fully billable for all insurance payors. Once finalized, the flow 200 may proceed to 220 where the user's initial assessment and plan may be completed and provided to the user, provided to medical billing systems for medical billing at 218, or a combination thereof. At 218, receiving electronic, written, or verbal objective information back from third parties may be key to proper and effective case management.

If, however, the physician assessment engine 208 determines that the digital record does need further review, the flow 200 may proceed to 212, where the physician assessment engine 208 may determine whether the user requires a face-to-face encounter with the physician. If the user is determined not to require a face-to-face encounter with the physician, the flow 200 may proceed to 214, where the flow 200 may include having the physician review and/or edit the digital record based on the information that the system 100 currently has. The digital record then may be finalized at 210 and signed off by the physician so that the flow 200 may proceed to 220, where the plan and assessment may be completed. If, however, at 212, the physician assessment engine 208 determines that the user requires a face-to-face encounter, the flow 200 may proceed to 216, where the user may either have a telemedicine encounter with the physician or may have an in-person visit at a facility that the physician works at. Based on the encounter, the physician may have additional information for the digital record and may either confirm the information in the digital record or edit/modify the information contained therein. Then, as with the other scenarios, the assessment and plan may be completed at 220 and provided to the user and to medical billing at 218.

Referring now also to FIG. 3, an exemplary process flow 300 for use with the system of FIG. 1 that facilitates updates to digital records of a patient, validates plans for patients, and facilitates medical billing according to embodiments of the present disclosure. At 302, updates to the digital record of the user needed to re-run the triage artificial intelligence engine 204 to generate a derivative digital record including a derivative note may be conducted. The update process may be iterative and may start with a master digital record plus new objective data filtered through the triage artificial intelligence engine 204 to produce a derivative digital record. Once filtered through the triage artificial intelligence engine 204, the flow 300 may conduct further updates to the digital record at 306. At 308, the flow may include conducting a variance analysis of the original digital record in comparison to the updated version of the digital record. In certain embodiments, the variance analysis of the digital record to the derivative digital record may be utilized to identify gaps and/or treatment plan changes between digital records. In certain embodiments, at 308, the physician may be enabled to addresses possible errors, new data, and make changes as needed and to publish a revised digital record including a revised S.O.A.P. note. In certain embodiments, the updates to the digital record may be looped back to the physician assessment engine 208 through certain rendering physicians may wish to manually review any changes to the original assessment and plan.

If there is no variance between the original digital record and the derivative/updated digital record, the flow 300 may proceed to 310 and validate the assessment and plan from the original digital record. If, however, there is a variance or discrepancy at 308, the flow 300 may proceed to 312. At 312, the physician may review to confirm the variance or edit/correct potential errors or inaccuracies. At 314, the flow 300 may include finalizing the digital record and obtaining the signature from the physician to complete the digital record. As a result, the flow 300 may proceed to 316, which results in the generation of a new assessment and plan for the user. Additionally, the digital record may be provided to a medical billing system at 218 for further review. The completed and signed digital record may be stored in long term storage at 316 and may be provided to the user to information and educate the user, as well as provide instructions to third parties on the method of care for the user.

At 318, the same user may conduct a new registration with the system 100 for another encounter with the system 100. At 320, the system 100 may determine if the encounter is a new case. If not, the flow 300 may proceed to obtain the digital records stored at 316 and provide them to the user and/or the physician for review. If, however, it is a new case/encounter for a new ailment, the flow 300 may proceed to utilizing the artificial intelligence triage engine 204, which may retrieve the saved digital record and then update the digital record for the new encounter and proceed through the steps of flows 200 and 300 as needed.

Notably, as shown in FIG. 1, the system 100 may perform any of the operative functions disclosed herein by utilizing the processing capabilities of server 160, the storage capacity of the database 155, or any other component of the system 100 to perform the operative functions disclosed herein. The server 160 may include one or more processors 162 that may be configured to process any of the various functions of the system 100. The processors 162 may be software, hardware, or a combination of hardware and software. Additionally, the server 160 may also include a memory 161, which stores instructions that the processors 162 may execute to perform various operations of the system 100. For example, the server 160 may assist in processing loads handled by the various devices in the system 100, such as, but not limited to, registering a user with the system 100; determining the user's chief medical complaint by utilizing the triage artificial intelligence engine 204; predicting likely assessment codes; generating digital records (e.g., S.O.A.P. notes), generating plans for a user based on analyzing user data, lab results, and other information; facilitating operative functionality of the physician assessment engine 208; updating digital records of a user; facilitating medical billing; and performing any other operations conducted in the system 100 or otherwise. In one embodiment, multiple servers 160 may be utilized to process the functions of the system 100. The server 160 and other devices in the system 100, may utilize the database 155 for storing data about the devices in the system 100 or any other information that is associated with the system 100. In one embodiment, multiple databases 155 may be utilized to store data in the system 100.

Although FIGS. 1-6 illustrate specific example configurations of the various components of the system 100, the system 100 may include any configuration of the components, which may include using a greater or lesser number of the components. For example, the system 100 is illustratively shown as including a first user device 102, a second user device 111, a communications network 135, a server 140, a server 145, a server 150, a server 160, and a database 155. However, the system 100 may include multiple first user devices 102, multiple second user devices 111, multiple communications networks 135, multiple servers 140, multiple servers 145, multiple servers 150, multiple servers 160, multiple databases 155, or any number of any of the other components inside or outside the system 100. Furthermore, in certain embodiments, substantial portions of the functionality and operations of the system 100 may be performed by other networks and systems that may be connected to system 100.

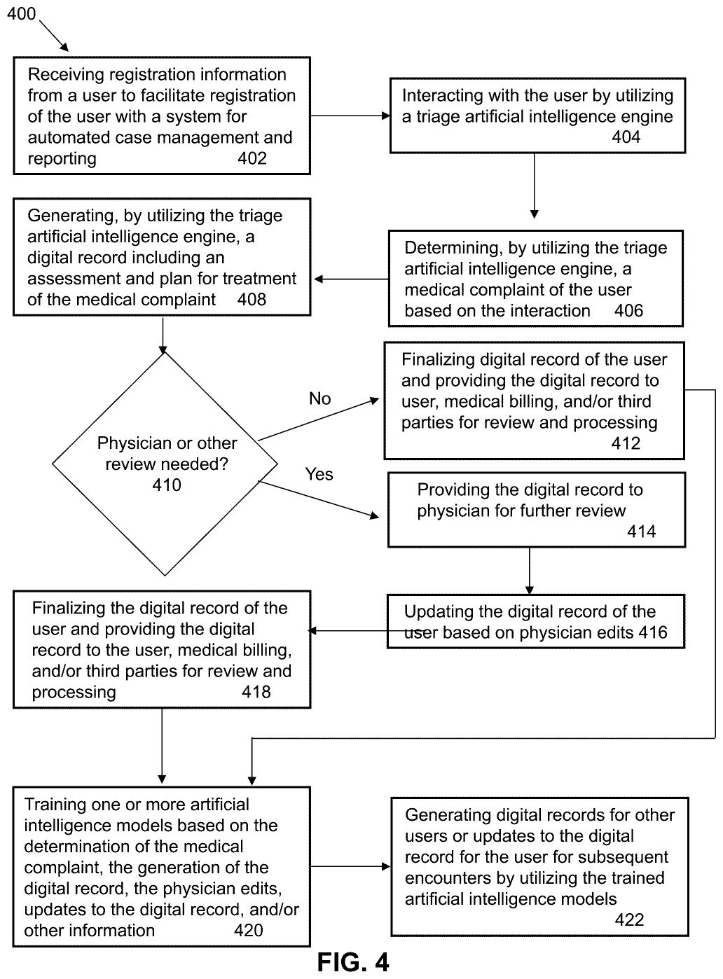

Notably, the system 100 may execute and/or conduct the functionality as described in the method(s) that follow. As shown in FIG. 4, an exemplary method 400 for providing an artificial intelligence system for providing automated case management and reporting is schematically illustrated. The method 400 and/or functionality and features supporting the method 400 may be conducted via an application of the system 100, devices of the system 100, processes of the system 100, any component of the system 100, or a combination thereof. The method 400 may include steps for obtaining data associated with a user (e.g., a patient), loading the data into artificial intelligence models for analysis, using the artificial intelligence models to compare the data to data utilized to train the artificial intelligence models, determining if the data associated with the user correlates and/or matches with data used to train the artificial intelligence models, generating predictions relating to a medical complaint of the user, an assessment associated with the user, a treatment plan for the user, or a combination thereof, and validating the predictions generating by the system 100, such as by physician review.

At step 402, the method 400 may include receiving information from a user (e.g., first user 101) to facilitate registration of the user with the system 100 for automated case management and reporting. For example, the user may input the information via a user interface of an application supporting the functionality of the system 100, such as via a first user device 102. The user may input information such as, but not limited to, demographic information, psychographic information, physiological information, payment information, health insurance information, any other information, or a combination thereof. Such information may include, but is not limited to, name, age, residence, current location, race, ethnicity, height, weight, eye color, skin color, body type, blood type, education level, income level, job title, credit card numbers, banking information, health insurance provider information (e.g., group number, individual number, etc.), mental state information, any other information, or a combination thereof. Additionally, in certain embodiments, the user may sign or otherwise consent (e.g., verbal authorization, biometric authorization, etc.) to consent forms that may be utilized to obtain consent from the user to receive treatment, examinations, and the like, such as from a physician (e.g., second user 110). In certain embodiments, the system 100 may also enable the user to create login credentials, which the user may utilized to authenticate into the application to access the system 100 and the user's information and/or digital records. In certain embodiments, the receiving of the information to register the user with the system 100 may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 404, the method 400 may include interacting with the user by utilizing a triage artificial intelligence engine of the system 100. In certain embodiments, the triage artificial intelligence engine may be configured to engage with the user, such as via the application supporting the functionality of the system 100. In certain embodiments, the triage artificial intelligence engine may interact with the user via voice, text, instant messaging, chat, any other digital interaction technology, or a combination thereof. For example, the triage artificial intelligence engine may ask voice-based or text-based questions to the user to facilitate identification of a medical complaint of the user based on the responses to the questions. The user, for example, may respond with an identification of symptoms that the user is experiencing, medical history information, the length of time of the symptoms, the intensity of the symptoms, any other information associated with a medical complaint, or a combination thereof. In certain embodiments, the information may be input by the user into the application and may include triage information. In certain embodiments, the interacting with the user can include utilizing cameras and/or sensor devices of the system 100 to capture media content (e.g., video content, audio content, audiovisual content, etc.) and/or sensor data of or otherwise associated with the user to determine additional information associated with the user. In certain embodiments, the cameras and/or sensor data can capture information and/or content associated with the user's skin, body, limbs, face, head, skin coloration, symptoms, organs, voice, movements, and/or other characteristics of the user to assist in determining a medical complaint and/or assessment of the user. The media content and/or sensor data can be utilized to supplement the information provided directly by the user into the system 100. At step 406, the method 400 may include having the triage artificial intelligence system analyze the information to identify and/or determine the medical complaint that the user is experiencing. In certain embodiments, the medical complaint may be determined by comparing the information obtained from the user to data utilized to train artificial intelligence models supporting the functionality of the triage artificial intelligence engine. In certain embodiments, media content and/or sensor data of the user can be compared to media content and/or sensor data used to train the artificial intelligence models supporting the functionality of the triage artificial intelligence engine. For example, the media content and/or sensor data utilized to train the models can be indicative of normal or healthy conditions (or conditions indicating sickness and/or disease) and can be compared to the media content and/or sensor data taken of the user to determine whether a specific medical complaint exists that may not necessarily have been determined from the information that the user directly input into the system 100.

In certain embodiments, the information from the user may be loaded into an artificial intelligence model(s) for analysis. In certain embodiments, artificial intelligence model(s) may be a file, program, module, and/or process that may be trained by the system 100 (or other system described herein) to recognize certain patterns, diagnoses, symptoms, behaviors, and/or content. For example, the artificial intelligence model(s) may be trained by the system 100 to detect specific types of objects, activity, occurrences, actions, motion, speed, and/or anything of interest. In certain embodiments, the artificial intelligence model may be, may include, and/or may utilize a Deep Convolutional Neural Network, a one-dimensional convolutional neural network, a two-dimensional convolutional neural network, a Long Short-Term Memory network, any type of machine learning system, any type of artificial intelligence system, or a combination thereof. In certain embodiments, the artificial intelligence model may incorporate the use of any type of artificial intelligence and/or machine learning algorithms to facilitate the operation of the artificial intelligence model(s). Notably, the system 100 may utilize any number of artificial intelligence models. The system 100 may train the artificial intelligence model(s) to reason and learn from data/information fed into the system 100 so that the model may generate and/or facilitate the generation of predictions about new data and information that is fed into the system 100 for analysis.

As an example, the artificial intelligence model(s) may be trained with data, such as, but not limited to, images, video content, audio content, text content, augmented reality content, virtual reality content, information relating to patterns, information relating to behaviors, information relating to characteristics of diseases, digital records containing patient data, sensor data (e.g., heart rate data, motion data, blood pressure data, oxygen data, blood glucose data, temperature data, etc.), any type of data, or a combination thereof. The data that is utilized to train the artificial intelligence model may be utilized by the artificial intelligence model to recognize diseases, medical conditions, psychological conditions, medical complaints (i.e., what the user is complaining about, such as foot pain, headache, stomach pain, etc.), or a combination thereof. For example, if the artificial intelligence model is trained with thousands of textual words that are known to be associated with the flu, the artificial intelligence model may learn that information that is fed into the model at a future time also are associated with the flu based on the future images and/or content having a correlation with the characteristics with any number of the textual words that are used to train the model. As additional data and/or content is fed into the model(s) over time, the model's ability to recognize medical conditions, complaints, and/or diseases will improve and will be more finely tuned. In certain embodiments, interactions with the user and/or the loading of the data/information into the artificial intelligence model(s) for analysis may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 408, the method 400 may include generating, by utilizing the triage artificial intelligence engine, a digital record for the user. In certain embodiments, the digital record may include a digital S.O.A.P note that may include subjective information relating to the observations relating to the symptoms provided by the user to the system 100, objective information (e.g., measurable data, such as vital signs, pulse rate, temperature, etc.), an assessment (e.g., a diagnosis of what the user is experiences, along with predicted assessment codes for the assessment), and a predicted plan for treating the diagnosis identified via the assessment. In certain embodiments, the generating of the digital record of the user may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 410, the method 400 may include determining whether physician or other review is needed of the digital record of the user, such as by utilizing a physician assessment engine including artificial intelligence models to determine what to do next with the digital record. If not, such as if there are standing order protocols that are already in place for the user's diagnosed condition, the method 410 may proceed to step 412. At step 412, the method 400 may include finalizing the digital record of the user and marking (or tagging) the digital record as complete. In certain embodiments, the physician may also sign the digital record to confirm completion. Additionally, at step 412, the finalized digital record may be provided to a medical billing system for billing purposes, third parties for review and/or processing, and/or to the user for the user's personal records. In certain embodiments, the finalizing of the digital record and/or the providing of the digital record to various parties may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

If, however, at step 410, it is determined by the system 100 that physician or other review is needed of the digital record (and/or the assessment, plan, etc.), the method 400 may proceed to step 414. At step 414, the method 400 may include providing the digital record to the physician for further review. In certain embodiments, the providing of the digital record may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. The physician may review the digital record and either confirm the information in the digital record or update/edit the digital record to correct the assessment, plan, and/or other information contained therein. Once the digital record is either confirmed or updated, the method 400 may proceed to step 418. At step 418, the method 400 may include finalizing the digital record of the user and providing the digital record to the user, to a medical billing system of the system 100 for billing purposes, and/or to third parties for review and/or processing.

At step 420, the method 400 may include training the artificial intelligence model(s) based on the determined medical complaint, the generation of the digital record, the physician edits, updates to the digital record, information provided by the user, predictions generated by the triage artificial intelligence engine, any information utilized by the system 100, or a combination thereof. In certain embodiments, the training may be configured to enhance predictions, deductions, reasoning, intelligence, correlations, outputs, analyses, and/or other capabilities of the artificial intelligence model(s). In certain embodiments, the training may be performed and/or facilitated by utilizing the first user 101, the second user 110 and/or by utilizing the first user device 102, the second user device 111, the server 140, the server 145, the server 150, the server 160, the communications network 135, any component of the system, any combination thereof, or by utilizing any other appropriate program, network, system, or device. At step 422, the method 400 may include generating digital records for other users, generating updates to the digital record for subsequent encounters by the user, or a combination thereof, by utilizing the trained artificial intelligence models supporting the functionality of the triage artificial intelligence engine, the physician assessment engine, the system 100 in general, or a combination thereof. In certain embodiments, the generating of the digital records may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. Notably, the method 400 may further incorporate any of the features and functionality described for the system 100, any other method disclosed herein, or as otherwise described herein.

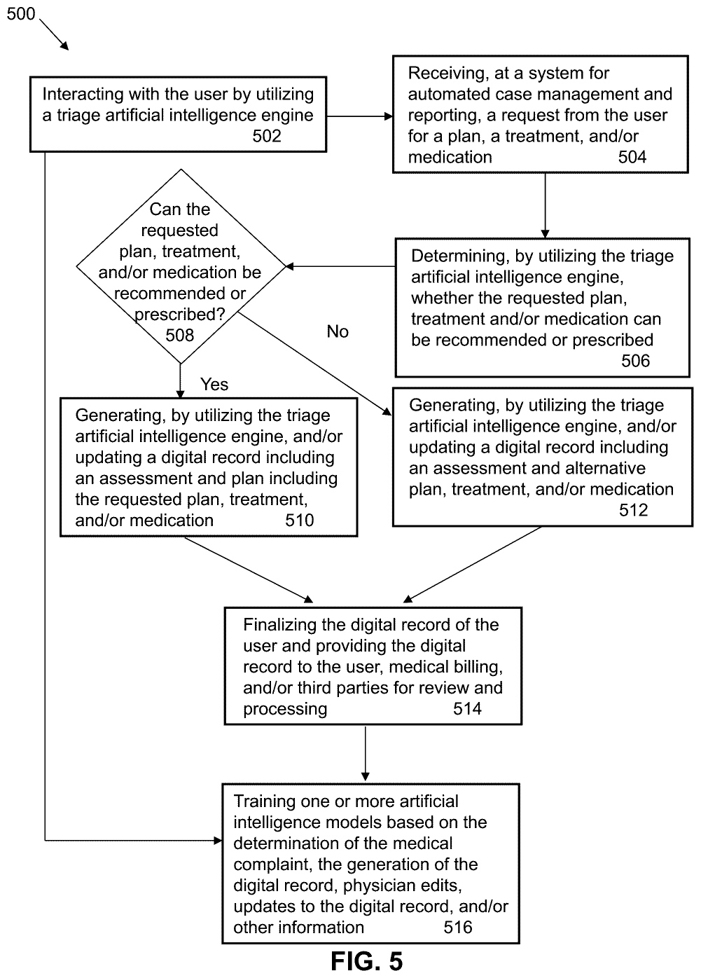

Notably, the system 100 may also execute and/or conduct the functionality as described in the method(s) that follow. As shown in FIG. 5, an exemplary method 500 for providing an artificial intelligence system including an inductive reasoning engine for enabling users to request specific plans, treatments, and/or medications is schematically illustrated. The method 500 and/or functionality and features supporting the method 500 may be conducted via an application of the system 100, devices of the system 100, processes of the system 100, any component of the system 100, or a combination thereof. The method 500 can include steps for enabling a user to request specific plans, treatments, and/or medications to a provider and/or to the automated case management and reporting platform (e.g., system 100), analyzing the request to determine if the requested plan, treatment and/or medication could harm the user or would not be recommended to the user, determining whether an insurance carrier can authorize the treatment, determining potential alternative plans, treatments, and/or medications, and training models based on the analyses and determinations enhance determinations of medical complains and determinations of assessments, plans, treatments, and/or medications to be recommended and/or prescribed to users.

At step 502, the method can include interacting with the user by utilizing a triage artificial intelligence engine. In certain embodiments, the interacting may include requesting information from the user, which the user can directly respond with answers and/or inputs. In certain embodiments, the interacting can include capturing media content of the user and/or sensor data associated with the user. For example, the interacting can include utilizing cameras to capture video of the user and/or sensors to capture all kinds of sensor data associated with the user (e.g., temperature, heart rate, blood pressure, sweat information, oxygen levels, blood information, glucose levels, etc.). In certain embodiments, the interaction with the user by the system 100 may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 504, the method 500 can include receiving, at the system 100, a request from the user for a specific plan, treatment, and/or medication, such as a plan treatment, and/or medication that the user wants for himself. In certain embodiments, the request can be received via the application of the system 100 and the receiving of the request may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. At step 506, the method 500 can include determining, such as by utilizing an inductive reasoning engine of the triage artificial intelligence engine (or other artificial intelligence engine of the system 100), to determine whether the requested plan, treatment, and/or medication can be recommended and/or prescribed to the patient. In certain embodiments, the determining can be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 508, the method 500 can include a decision step based on whether the requested plan, treatment and/or medication can be recommended or prescribed to the user. In certain embodiments, the determining may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. The determining as to whether the requested plan, treatment, and/or medication can be prescribed can include analyzing an existing digital record of the user that may include a medical history, a list of medication being taken by the user, a list of conditions experienced in the past and/or present by the user, a list of allergies, and/or any other information associated with the user, along with medical literature and information on diseases, medications, treatments, and/or assessments. If the requested plan, treatment, and/or medication would harm the user, would not be recommended to the user (e.g., other treatments and/or medications may be preferred for the particular medical complaint), would not be approved by an insurance carrier, and/or is not medically necessary for the user for the particular medical complaint, or a combination thereof, the method 500 can determine that the requested plan, medication, and/or treatment should not be provided to the user.

If the requested plan, treatment, and/or medication can be recommended or prescribed based on the analysis by the system 100, the method 500 can proceed to step 510. At step 510, the method 500 can include generating, such as by utilizing the indicative reasoning engine (e.g., a software engine or combination of software and hardware engine), and/or updating a digital record for the user including an assessment for the user and the requested plan, treatment and/or medication. In certain embodiments, the generating and/or updating may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. If, however, the requested plan, treatment, and/or medication cannot be recommended or prescribed to the user (e.g., such as if there is a contraindication, conflict, medically not necessary, not approved by insurance carrier, etc.), the method 500 can proceed to step 512, which can include generating and/or updating a digital record of the user that includes an assessment and an alternative plan, treatment, and/or medication for recommendation for the user. In certain embodiments, the generating and/or updating of the digital record may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 514, the method 500 can include finalizing the digital record of the user and providing the digital record to the user, medical billing, and/or third parties for review and processing. In certain embodiments, at step 514, a provider can edit and/or approve the generated record as well. At step 516, the method can include training one or more artificial intelligence models based on the determination of the user's medical complaint, the determinations, the generating and/or updating of the digital record, provider edits, and/or other information to facilitate enhancement of future determinations for the same user and/or other users that interact with the system. IN certain embodiments, the training may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. Notably, the method 500 may be repeated as necessary and may further incorporate any of the features and functionality described for the system 100, any other method disclosed herein, or as otherwise described herein.

The systems and methods disclosed herein may include still further functionality and features. For example, the operative functions of the system 100 and method may be configured to execute on a special-purpose processor specifically configured to carry out the operations provided by the system 100 and method. Notably, the operative features and functionality provided by the system 100 and method may increase the efficiency of computing devices that are being utilized to facilitate the functionality provided by the system 100 and the various methods discloses herein. For example, by training the system 100 over time based on data and/or other information provided and/or generated in the system 100, a reduced amount of computer operations may need to be performed by the devices in the system 100 using the processors and memories of the system 100 than compared to traditional methodologies. In such a context, less processing power needs to be utilized because the processors and memories do not need to be dedicated for processing. As a result, there are substantial savings in the usage of computer resources by utilizing the software, techniques, and algorithms provided in the present disclosure. In certain embodiments, various operative functionality of the system 100 may be configured to execute on one or more graphics processors and/or application specific integrated processors.

Notably, in certain embodiments, various functions and features of the system 100 and methods may operate without any human intervention and may be conducted entirely by computing devices. In certain embodiments, for example, numerous computing devices may interact with devices of the system 100 to provide the functionality supported by the system 100. Additionally, in certain embodiments, the computing devices of the system 100 may operate continuously and without human intervention to reduce the possibility of errors being introduced into the system 100. In certain embodiments, the system 100 and methods may also provide effective computing resource management by utilizing the features and functions described in the present disclosure. For example, in certain embodiments, devices in the system 100 may transmit signals indicating that only a specific quantity of computer processor resources (e.g. processor clock cycles, processor speed, etc.) may be devoted to training the artificial intelligence model(s), comparing information obtained from a patient to information contained in and/or used by the artificial intelligence model(s), determining whether information correlates with information and/or content utilized to train an artificial intelligence model(s), generating predictions relating to plans, medical complaints, diagnoses, and/or other predictions, facilitating output of responses based on various conditions, and/or performing any other operation conducted by the system 100, or any combination thereof. For example, the signal may indicate a number of processor cycles of a processor may be utilized to update and/or train an artificial intelligence model, and/or specify a selected amount of processing power that may be dedicated to generating or any of the operations performed by the system 100. In certain embodiments, a signal indicating the specific amount of computer processor resources or computer memory resources to be utilized for performing an operation of the system 100 may be transmitted from the first and/or second user devices 102, 111 to the various components of the system 100.

In certain embodiments, any device in the system 100 may transmit a signal to a memory device to cause the memory device to only dedicate a selected amount of memory resources to the various operations of the system 100. In certain embodiments, the system 100 and methods may also include transmitting signals to processors and memories to only perform the operative functions of the system 100 and methods at time periods when usage of processing resources and/or memory resources in the system 100 is at a selected value. In certain embodiments, the system 100 and methods may include transmitting signals to the memory devices utilized in the system 100, which indicate which specific sections of the memory should be utilized to store any of the data utilized or generated by the system 100. Notably, the signals transmitted to the processors and memories may be utilized to optimize the usage of computing resources while executing the operations conducted by the system 100. As a result, such functionality provides substantial operational efficiencies and improvements over existing technologies.

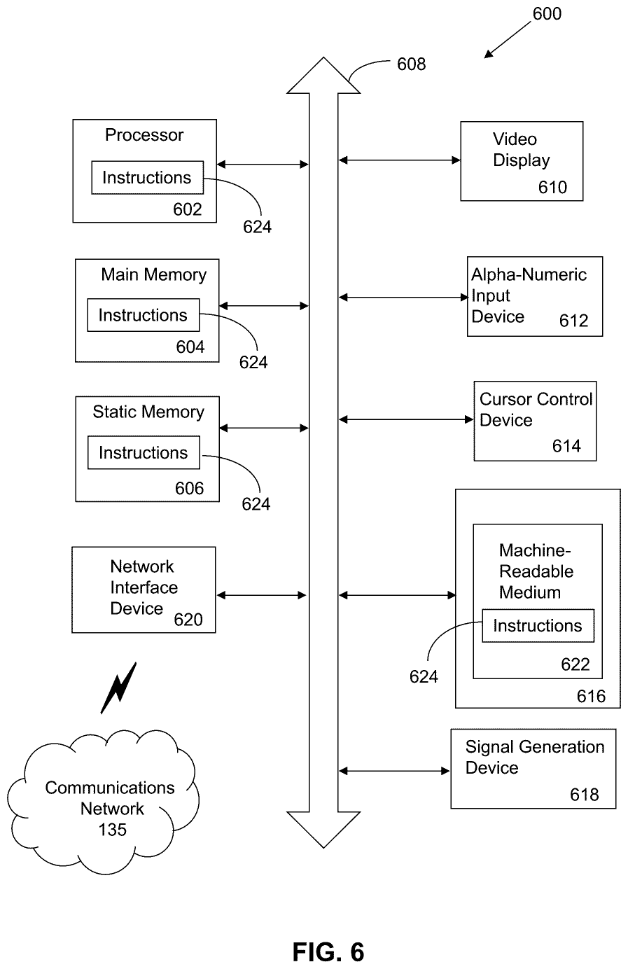

Referring now also to FIG. 6, at least a portion of the methodologies and techniques described with respect to the exemplary embodiments of the system 100 can incorporate a machine, such as, but not limited to, computer system 600, or other computing device within which a set of instructions, when executed, may cause the machine to perform any one or more of the methodologies or functions discussed above. The machine may be configured to facilitate various operations conducted by the system 100. For example, the machine may be configured to, but is not limited to, assist the system 100 by providing processing power to assist with processing loads experienced in the system 100, by providing storage capacity for storing instructions or data traversing the system 100, or by assisting with any other operations conducted by or within the system 100. As another example, the computer system 600 may assist with generating models associated with generating predictions relating to a diagnosis of a patient (e.g., first user 101), predictions relating to identification of a medical complaint of the patient, predictions relating to changes in a physical or mental condition of the patient over time, any type of predictions generated by the system 100, or a combination thereof. As another example, the computer system 600 may assist with generating assessments of the patient, interacting with the patient (e.g., by transmitting questions to the first user 101, supporting a teleconference for a patient visit, any other interactions, or a combination thereof), registering the patient with the system 100, conducting medical billing, facilitating execution of physician orders for the patient, any other functionality provided by the system 100, or a combination thereof.

In certain embodiments, the machine may operate as a standalone device. In some embodiments, the machine may be connected (e.g., using communications network 135, another network, or a combination thereof) to and assist with operations performed by other machines and systems, such as, but not limited to, the first user device 102, the second user device 111, the server 140, the server 145, the server 150, the database 155, the server 160, any other system, program, and/or device, or any combination thereof. The machine may be connected with any component in the system 100. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 600 may include a processor 602 (e.g., a central processing unit (CPU), a graphics processing unit (GPU, or both), a main memory 604 and a static memory 606, which communicate with each other via a bus 608. The computer system 600 may further include a video display unit 510, which may be, but is not limited to, a liquid crystal display (LCD), a flat panel, a solid state display, or a cathode ray tube (CRT). The computer system 600 may include an input device 612, such as, but not limited to, a keyboard, a cursor control device 614, such as, but not limited to, a mouse, a disk drive unit 616, a signal generation device 618, such as, but not limited to, a speaker or remote control, and a network interface device 620.

In certain embodiments, the disk drive unit 616 may include a machine-readable medium 1022 on which is stored one or more sets of instructions 624, such as, but not limited to, software embodying any one or more of the methodologies or functions described herein, including those methods illustrated above. The instructions 624 may also reside, completely or at least partially, within the main memory 604, the static memory 606, or within the processor 602, or a combination thereof, during execution thereof by the computer system 500. In certain embodiments, the main memory 604 and the processor 602 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 622 containing instructions 624 so that a device connected to the communications network 135, another network, or a combination thereof, can send or receive voice, video or data, and communicate over the communications network 135, another network, or a combination thereof, using the instructions. The instructions 624 may further be transmitted or received over the communications network 135, another network, or a combination thereof, via the network interface device 620.

While the machine-readable medium 622 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. In certain embodiments, the “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure is not limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

We claim:

1. A system, comprising:
a memory that stores instructions; and
a processor configured to execute the instructions to:
register, via an interface of the system, an individual with the system;
interact, by utilizing a triage artificial intelligence engine, with the individual to obtain information from the individual;
identify, by utilizing the triage artificial intelligence engine and based on the information, a medical complaint associated with the individual, wherein the medical complaint is identified based on the information having a correlation with medical complaint information utilized to train the triage artificial intelligence engine;
generate, by utilizing the triage artificial intelligence engine and based on the medical complaint, a digital record associated with the individual, wherein the digital record includes a plan associated with addressing the medical complaint;
finalize the digital record if a standing order protocol exists that matches criteria associated with the plan in the digital record;
provide, if the standing order protocol does not exist, the digital record for further review by a provider; and
modifying, based on an input received that is associated with the further review, the digital record.

2. The system of claim 1, wherein the processor is further configured to generate the plan based on the information, the medical complaint, or a combination thereof, correlating with at least one plan utilized to train the triage artificial intelligence engine.

3. The system of claim 1, wherein the processor is further configured to register the individual with the system based on receiving registration information associated with the individual via an interface of the system.

4. The system of claim 1, wherein the processor is further configured to determine whether the individual requires a consultation with the provider.

5. The system of claim 4, wherein the processor is further configured to provide the digital record including the plan to the provider via a worklist for the provider if the consultation with the provider is not required.

6. The system of claim 4, wherein the processor is further configured to facilitate a teleconference for the consultation between the individual and the provider if the individual is determined to require the consultation with the provider.

7. The system of claim 6, wherein the input associated with the further review is generated based on the teleconference for the consultation between the individual and the provider.

8. The system of claim 7, wherein the processor is further configured to finalize the digital record based on the input by updating the plan based on additional information obtained via the teleconference for the consultation.

9. The system of claim 1, wherein the processor is further configured to train an artificial intelligence model using training information utilized to facilitate identification of medical complaints, generation of plans, identification of diagnoses associated with the medical complaint, or a combination thereof.

10. The system of claim 1, wherein the processor is further configured to transmit the digital record to a medical billing system for further processing after the digital record is finalized.

11. The system of claim 1, wherein the processor is further configured to facilitate initiation of a treatment, a procedure, dispensing of a medication, a medical test, or a combination thereof, in accordance with the plan of the digital record.

12. The system of claim 1, wherein the processor is further configured to generate a derivative digital record based on updates to the information that occur over time.

13. The system of claim 12, wherein the processor is further configured to conduct a variance analysis of the digital record with the derivative digital record to detect a discrepancy between the digital record and the derivative record, a change in the plan of the digital record and a derivative plan in the derivative digital record, occurrence of an error, or a combination thereof.

14. A method, comprising:
interacting, by utilizing a triage artificial intelligence engine of a system, with an individual to obtain information from the individual during a first encounter with the individual;
identifying, by utilizing the triage artificial intelligence engine and based on the information, a medical complaint associated with the individual, wherein the medical complaint is identified based on the information having a correlation with medical complaint information utilized to train the triage artificial intelligence engine;
generating, by utilizing the triage artificial intelligence engine and based on the medical complaint, a digital record associated with the individual, wherein the digital record includes a plan associated with addressing the medical complaint;
finalizing the digital record if a standing order protocol exists that matches criteria associated with the plan in the digital record;
providing, if the standing order protocol does not exist, the digital record for further review by a provider; and
modifying, based on an input received that is associated with the further review, the digital record.

15. The method of claim 14, further comprising registering, via an interface of the system, the individual with the system.

16. The method of claim 14, further comprising determining whether a second encounter with the individual is associated with the medical complaint.

17. The method of claim 16, further comprising presenting the digital record associated with the individual to the individual to confirm whether a change is to be made to the digital record if the second encounter is associated with the medical complaint.

18. The method of claim 16, further comprising generating a new digital record if the second encounter is not associated with the medical complaint.

19. The method of claim 14, further comprising generating the plan for the digital record based on an assessment of the individual, subjective data associated with the individual, objective data associated with the individual, or a combination thereof.

20. A non-transitory computer-readable device comprising instructions, which, when loaded and executed by a processor, cause the processor to perform operations, the operations comprising:
interacting, by utilizing a triage artificial intelligence engine of a system, with an individual to obtain information from the individual during a first encounter with the individual;
identifying, by utilizing the triage artificial intelligence engine and based on the information, a medical complaint associated with the individual, wherein the medical complaint is identified based on the information having a correlation with medical complaint information utilized to train the triage artificial intelligence engine;
generating, by utilizing the triage artificial intelligence engine and based on the medical complaint, a digital record associated with the individual, wherein the digital record includes a plan associated with addressing the medical complaint;
finalizing the digital record if a standing order protocol exists that matches criteria associated with the plan in the digital record;
providing, if the standing order protocol does not exist, the digital record for further review by a provider; and
modifying, based on an input received that is associated with the further review, the digital record.
